(19) United States
(12) Plant Patent Application Publication
(10) Pub. No.: US 2008/0155724 P1
(43) Pub. Date: Jun. 26, 2008

(54) Crepemyrtle Tree Named 'Cascading Pink Lady'

(76) Inventors: Robert E. Hambuchen, Conway, AR (US); Betty J. Hambuchen, Conway, AR (US)

Correspondence Address: ROBERT E. HAMBUCHEN, 3040 DALLAS LOOP, CONWAY, AR, 72034, US

(21) Appl. No.: 11/645,310

(22) Filed: Dec. 26, 2006

(51) Int. Cl. A01H 5/00

(52) U.S. Cl. PLT/252

(57) ABSTRACT

Cascading Pink Lady is a new and distinct variety of Lagerstroemia Indica Prostrata, commonly referred to as Crepemyrtle. It is a low growing variety where the primary limbs cascade out and downward while producing multiple secondary along the primaries. It reaches a height of 16 to 20 inches at maturity with substantially no upward growth. It can reach a width of 36 to 40 inches. This cultivar blooms in mid June in Zone 7B and normally is in full bloom by the end of June. Because of its bloom habit it sometimes appears to be a multi-colored Crepemyrtle. While most new blooms are a Red Purple 72D a few blooms are very lite in color, with some appearing almost white. The plant is very resistant to powdery mildew and is cold hardy with no visible damage at 8 degrees Farenheit.

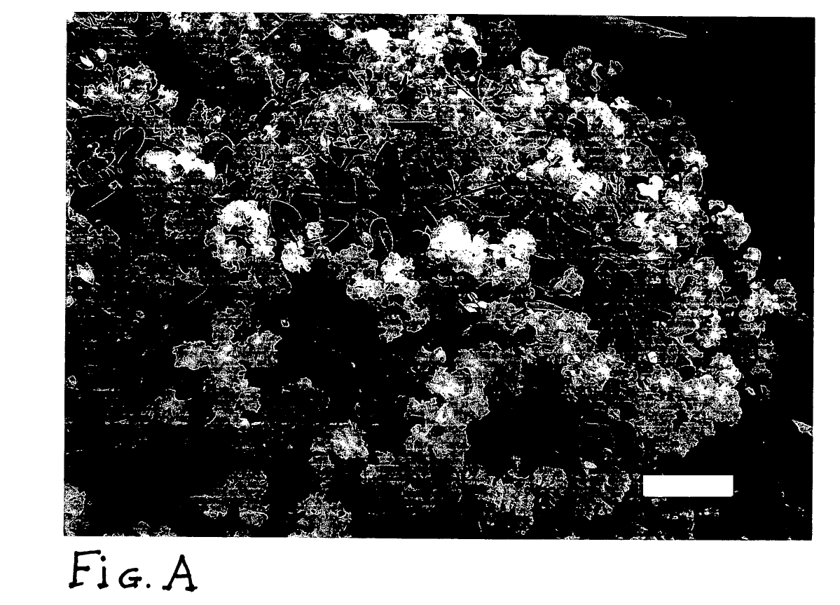

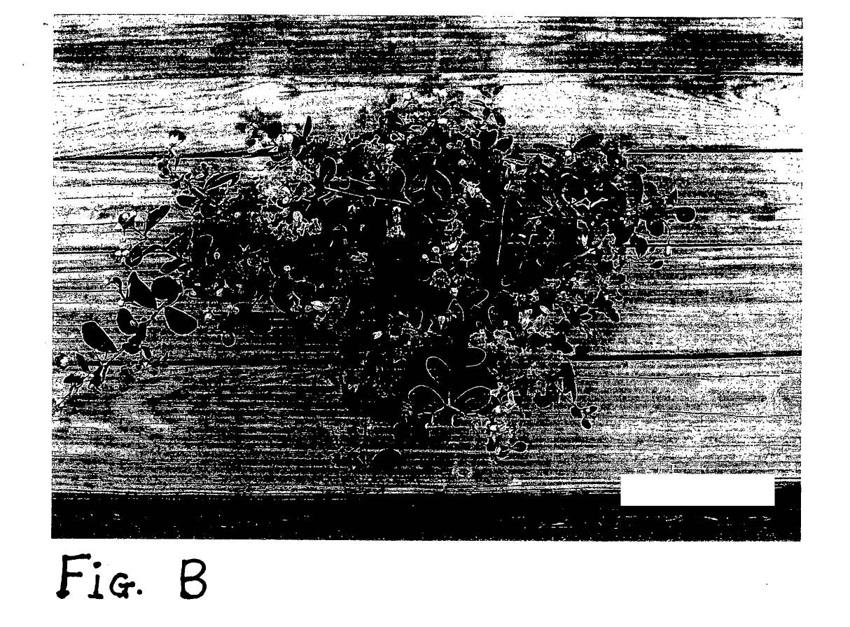

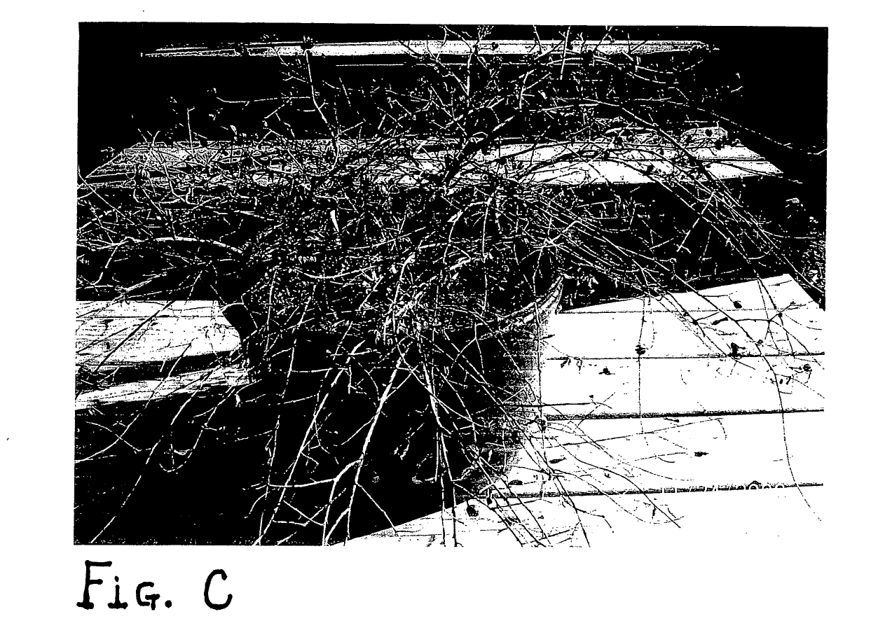

### CROSS REFERENCE TO RELATED APPLICATIONS

Not applicable

### STATEMENT REGARDING FEDERALLY-SPONSORED RESEARCH AND DEVELOPMENT

Not applicable

### LATIN NAME OF THE GENUS AND SPECIES OF THE PLANT CLAIMED

Lagerstroemia Indica “Prostrata”

### VARIETY DENOMINATION

Lagerstroemia Indica “Cascading Pink Lady”, Commonly known as Crepe Myrtle.

### BACKGROUND OF THE INVENTION

1. Field of Invention

The present invention relates to a new and a very distinct variety of landscape plant, Lagerstroemia Prostrata “Cascading Pink Lady”, commonly known as Crepemyrtle.

2. Description of Prior Art Including Information Disclosed Uinder 37 CFR 1,97 and 1.99.

This new and aforementioned cultivar, Cascading Pink Lady is a whole plant sport of Rosey Carpet PP 13965. It was found growing in our cutting bed facility. This new cultivar is a low growing and spreading plant with a cascading growth pattern. This new plant has been propagated from both hardwood and soft wood cuttings over several generations. Each generation of cuttings produce plants identical to the original seedling plant.

### SUMMARY OF INVENTION

This invention involves the discovery of a new and distinct variety of Crepemyrtle, Lagerstroemia Indica “Prostrata” which we have named Cascading Pink Lady. It is characterized as a vigorous, low growing and spreading cultivar with arching or cascading limbs. The plant reaches a height of 16 to 20 inches at maturity with substantially no vertical growth. Shortly after the primary limbs develop and grow we start to see secondary limbs develop. During bloom season each secondary produces an inflorescence. This provides color over the entire plant. The stamens on all blooms retain their yellow color well into the life of the bloom but fade and dry up before the bloom falls. In a normal year Cascading Pink Lady starts to bloom in mid June and is in full bloom by the end of June in Central Arkansas, zone 7B. Plants continue to bloom well into October. The Cultivar produces seed but not in heavy or excessive amounts.

### BRIEF DESCRIPTION OF THE DRAWINGS

FIG. A Close up and partial photo of Cascading Pink Lady in full bloom showing the color variation as the blooms start to mature.

FIG. B Recently propagated 3^{rd} generation Cascading Pink Lady

FIG. C Picture of the defoliated plant more clearly showing its natural structure.

### DETAILED BOTANICAL DESCRIPTION OF THE PLANT

This new variety of Crepemyrtle, Lagerstroemia Indica “Prostrata”, named Cascading Pink Lady is a whole plant sport of Rosey Carpet PP13965. This is a small compact plant with arching or cascading limbs.

The cultivar has been reproduced over several generations and it remains identical to the original seedling plant.

Each plant produces a number of secondary branches with inflorescences along each of the primary branches. It is not unusual to see up to 21 rather small inflorescences per primary limb including those on the secondary limbs. They, the inflorescences, range in size from 6 to 10 centimeters in length with a width of 4 to 8 centimeters.

This new cultivar reaches a height of 16 to 20 inches at maturity with substantially no vertical growth. In our growing area the plant reaches 36 to 40 inches in width. Old limbs retain their cascading form. However we have never seen any sign of defoliating bark.

Color of the tops of old growth mature limbs is Brown Group 200D, the Bottom or underside of these same limbs, are a Grey Brown 190B. The color of the tops of new growth limbs are Red Purple Group 59B. The edges of these new growth limbs carry this same 59B color along each side with a color of Yellow Green 146B on the very bottom. [Please note, these colors and all other colors used to describe all parts of this plant, in this application, are taken from The Royal Horticultural Society colour Charts.]

Most often the leaf arrangement on Crepe Myrtle Cascading Pink Lady are alternate with an occasional opposite arrangement. Leaves are ellipse or ovate with an acute tip and base. Mature leaves are relatively small in size. They vary in length of 2.7 to 3.2 centimeters and in width from 1.6 to 2.1 centimeters. Mature leaf color is a Green 137A on the Adaxil side and 137D on the abaxil, the leaf edge is also a 137D. New early growth leaves on the adaxil side are Green group 137B, the abaxil side is a Green Group 137D, edges are also a Green Group 137D with the stem or axis a Green Group 137C. Then as the leaves mature this color changes to the 137A. Bloom buds on Cascading Pink Lady are 0.5 to 0.7 cm in width and 0.7 to 1.0 cm in length. Bud color is a mostly a 145C with a few 145D with the bottom of the bud a 145B. Sepals are 0.3 cm in width and 0.9 to 1.0 cm in length. In late summer about 1 in 10 bloom buds will have a noticeable small black dot, about the size of a pin point about a 0.2 cm from the top and along the edges of where the edge of two sepals meet.

Blooms open in mid June and are in full color by June 30^{th} in Zone 7B. It continues to bloom well into October. Flower color, including the edges opens with a Red Purple Group 72D, it soon fades to a 70C and then to a 70D. And occasionally the plant produces a newly opened bloom of 70C, its also not unusual to see a new bud open a 70D [both in the Red Purple Group.] This pattern of bloom color change sometimes gives the appearance of a multi-colored Crepemyrtle.

This Crepemyrtle is a very cold hardy plant having survived with no damage at 8 degrees Fahrenheit. Also it is highly resistant to powdery mildew. No powdery mildew has ever been observed on the cultivar in our growing area.

1. We claim a new and a distinct variety of crepemyrtle plant substantially as described and shown, characterized by a low growing with extensive cascading lateral branching that reaches a mature height of 16 to 20 inches, with substantially no upward growth, with profuse and distinctive soft pink inflorescences that, in a consistent pattern, tend to fade as they begin to mature and this new plant has a very high resistant to powdery mildew.
